(19) United States
(12) Patent Application Publication
(10) Pub. No.: US 2011/0206770 A1
(43) Pub. Date: Aug. 25, 2011

(54) ATOVAQUONE WITH A PARTICLE SIZE DIAMETER RANGE (D90) OF GREATER THAN 3 MICRONS TO ABOUT 10 MICRONS

(75) Inventors: Brett Antony Mooney, Queensland (AU); Panaglotis Keramidas, Queensland (AU)

(73) Assignee: Alphapharm Pty. Ltd., Carole Park, Queensland (AU)

(21) Appl. No.: 13/054,888

(22) PCT Filed: Feb. 4, 2009
(86) PCT No.: PCT/AU2009/000133

§ 371 Date: Mar. 17, 2011

(30) Foreign Application Priority Data
Jul. 25, 2008 (AU) 2008903802

(51) Int. Cl. A61K 9/14; C07C 279/26; A61K 31/122; A61K 31/155; A61P 33/06

(52) U.S. Cl. 424/489; 428/402; 514/681; 514/635; 568/309

(57) ABSTRACT

Atovaquone or a pharmaceutically acceptable salt thereof having a particle size diameter range with a D₉₀ of between greater than 3 to about 10 μm.

### TECHNICAL FIELD

The present invention relates to pharmaceutical compound comprising atovaquone having a particular particle size and compositions comprising such atovaquone as the active ingredient. Further, the invention relates to the use of such compositions in the manufacture of medicaments for the treatment of conditions for which atovaquone is effective and processes to manufacture these compositions.

### BACKGROUND ART

The compound trans-2-[4-(4-chlorophenyl)cyclohexyl]-3-hydroxy-1,4-naphthalenedione, hereafter referred to as atovaquone was first disclosed in U.S. Pat. No. 5,053,432 (assigned to Welcome Foundation). Atovaquone is an antipneumocystic agent having the structure:

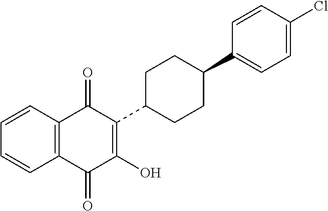

Atovaquone is indicated for the prophylaxis of Plasmodium falciparum malaria and the treatment of acute, uncomplicated P. falciparum malaria.

Atovaquone is approved for marketing in the US under the tradename Mepron® as tablets of 250 mg and an oral suspension. It is also available in combination with proguanil hydrochloride under the tradename Malarone®. These products are oral tablets in 250 mg/100 mg and 62.5 mg/25 mg strengths of the atovaquone/proguanil HCl, respectively.

Proguanil has the chemical name N-(4-chlorophenyl)-N′-(1-methylethyl)imidodicarbonimidic diamide. It is an antiprotozoal agent having the structure:

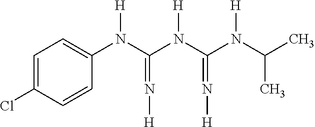

Proguanil also has the chemical name 1-(4-chlorophenyl)-5-isopropyl-biguanide. It is commercially available as a hydrochloride salt. Proguanil is an antiprotozoal drug. Other antiprotozoal drugs include cycloguanil, mefloquinine, quinine, amodiaquine, chloroquine, hydroxychloroquine, pamaquine, primaquine, pyrimethamine, artemisinin, artemether, artesunate, artenimol, artemotil, halofantrine, lumefantrine and pharmaceutically acceptable salts thereof.

Pharmaceutically active substances are commonly formulated into dosage forms to aid the delivery of small amounts thereof. The amount of pharmaceutically active substance that will be present in oral dosage forms can vary from a very small amount such as about 0.05 mg up to larger amounts such as about 1000 mg, depending on the pharmaceutically active substance being used and the therapeutic and/or prophylactic effective amount thereof. In order to be able to accurately administer these amounts of pharmaceutically active substance, the oral dosage form is often constituted of other pharmaceutically acceptable excipients that perform various functions depending on the dosage form and the mode of action required. These excipients have an effect on the method and rate of delivery of the pharmaceutically active substance to the patient.

Another aspect of pharmaceutical formulations that affects the rate of delivery or the bioavailability of the pharmaceutically active substance is the particle size thereof. This relationship between particle size and bioavailability is well known in the pharmaceutical industry and across a range of pharmaceutical products. In 1979, studies into the effect of crystal size on the bioavailability of Benoxaprofen were conducted (Biomed Mass Spectrom., 1979 April, 6(4), pp 173-8, Wolen R L et al; J. Pharm. Sci., 1979 July, 68(7), pp 850-2, Ridolfo A S et al). J. Pharm. Sci., 1980 April, 69(4), pp 391-4, Schoenwald R D & Stewart P disclose the effect of particle size on the ophthalmic bioavailability of dexamethasone stating that “A statistically significant rank-order correlation was observed between increasing drug levels and decreasing particle size.” Other examples include American Journal of Veterinary Research, 1980 December, 41(12), pp 2095-2101, Shastri S et al; Clinical Pharmacokinetics, 1998 February, 34(2), pp 155-62, Miller D B & Spence J D; Current Med Res Opin, 2000, 16(2), pp 134-8, Guichard J P et al; J. Microencapsul., 2001 May-June, 18(3), pp 359-71, Demirel M et al; and Pharmaceutical Dev Technol, 2004, 9(1), pp 1-13, Rasenick N & Muller B W. Also refer to U.S. Patent Application No. 2002/0035119 Al Rajiv, M et al; U.S. Patent Application No. 2003/0175338 Al Manoj, K P et al; WO 03/082241 A3 Kumar, P M et al; WO 03/080056 A2 (Teva Pharmaceutical Industries Ltd); and U.S. Reissue Pat. No. RE37516 E Grebow, P E et al that discuss the relationship between particle size and bioavailability of the pharmaceutically active substance.

WO 98/35681 (Novartis) illustrates the effect of reducing the particle size of a drug with poor aqueous solubility. The formulations disclosed therein comprise micronised oxcarbazepine particles with a median particle size of between 2-12 microns (μm). Such particle size enhances the dissolution rate and consequently the bioavailability. The problem with micronised particles of such a small size is that the particles can agglomerate into larger particles, thereby reducing the solubility and consequently the bioavailability of the drug. Also micronising to a small particle size can also lead to stability and/or discolouration problems. Additionally, micronisation to such a small particle size requires greater energy input, more time and greater controls on the micronisation process to achieve the required range whilst reducing the amount of rejected material.

The problem with compositions comprising small particle sizes is that there are some instances in which particle size reduction fails to increase absorption rate. One reason might be that dissolution is not the rate limiting step. Additionally, micronisation can sometimes increase the tendency of the particles to aggregate which may lead to a decrease in surface area. Further it has been reported that extremely small sizes may be inadvisable for some drug substances as adsorbed air or crystal growth might act as dissolution rate limiting steps. Thus, to sum up the effect on absorption behaviour for particle size reduction to extremely small particles less than 10 microns cannot be reliably predicted. The micronisation process itself can also lead to degradation of the active ingredient.

Conversely, relatively larger particle sizes of drugs that have low aqueous solubility can suffer from the problem of poor dissolution and consequently poor bioavailability. Thus, there is a need for compositions of atovaquone to provide improved or effective compositions that keep the beneficial properties of micronised particles, such as an increase in aqueous solubility, leading to an increase in bioavailability whilst overcoming the above highlighted problems of the prior art.

U.S. Pat. Nos. 6,018,080 and 6,649,659 disclose processes for the production of microfluidised particles of atovaquone. The atovaquone is mixed with a liquid vehicle at a concentration of less than 450 mg/mL. This mixture is subjected to at least 3 passes through a Microfluidiser, preferably to achieve a particle size whereby at least 90% of the particles have a volume diameter between 0.1 to 3 μm. The efficacy of atovaquone as a therapeutic agent is limited by its bioavailability. Accordingly, the invention of this prior art was to provide atovaquone in a more bioavailable form.

Additionally, these patents disclose that the bioavailability of atovaquone can be increased by ensuring that the particle size is within a certain defined range of small particles. However, the teaching therein is that conventional methods of reducing the particle size of atovaquone were found to be unsuccessful in producing particles of the size required to improve bioavailability.

### SUMMARY OF INVENTION

The inventors have surprisingly found that, against the teaching of the prior art, a pharmaceutical composition comprising atovaquone of a defined particle size affords suitable properties which overcome the above problems associated with the prior art. A pharmaceutical composition containing such atovaquone surprisingly exhibits bioequivalency to atovaquone of the prior art. Such atovaquone may be prepared by conventional methods of particle size reduction.

The defined range disclosed in the prior art is between 0.1 to 3 microns, thus the prior art teaches that bioavailability is poor outside this range, however against the prior art teaching the inventors have surprisingly found that ranges between greater than 3 to about 10 microns are just as bioavailable and can be prepared by conventional methods without the added cost and processing to produce such small particles.

Accordingly there is provided in a first aspect of the invention particulate atovaquone or a pharmaceutically acceptable salt thereof having a D₉₀ particle size of greater than 3 to about 10 microns. In an embodiment the D₉₀ particle size diameter is greater than 3 to about 5 microns. In an embodiment the D₉₀ particle size is about 4 to about 5 microns. In an alternative embodiment the D₉₀ particle size diameter is about 6 to about 8 microns.

In a second aspect of the invention there is provided a pharmaceutical composition comprising atovaquone or a pharmaceutically acceptable salt thereof having a D₉₀ particle size of greater than 3 to about 10 microns and further comprising one or more pharmaceutically acceptable excipients. In an embodiment the D₉₀ particle size diameter is greater than 3 to about 5 microns. In an embodiment the D₉₀ particle size is about 4 to about 5 microns. In an alternative embodiment the D₉₀ particle size diameter is about 6 to about 8 microns.

A composition according to the invention may be a tablet composition. The tablet may be coated. In an alternative embodiment the composition is a capsule.

In an embodiment the composition comprises atovaquone or a pharmaceutically acceptable salt thereof present in an amount of approximately 1-90% by weight of the composition.

Further embodiments of a composition according to the invention further comprise wetting agents/surfactants, such as Tween® (polysorbate) or sodium lauryl sulphate.

The pharmaceutical composition of the second aspect of the invention can optionally include one or more additional API's. Preferably the API's are selected from the group comprising antiprotozoal agents.

In a third aspect of the invention there is provided the use of atovaquone or a pharmaceutically acceptable salt thereof having a D₉₀ particle of greater than 30 to about 10 microns in the manufacture of a medicament for the prophylaxis and/or treatment of malaria.

In a fourth aspect of the invention there is provided the use of atovaquone or a pharmaceutically acceptable salt thereof having a D₉₀ particle size of greater than 3 to about 10 microns or a pharmaceutical composition comprising said atovaquone or a pharmaceutically acceptable salt thereof for the prophylaxis and/or treatment of malaria.

In a fifth aspect of the invention there is provided a method for the treatment or prophylaxis of malaria comprising administering to a subject a pharmaceutical composition comprising atovaquone or a pharmaceutically acceptable salt thereof having a D₉₀ particle size of greater than 3 to about 10 microns.

In a sixth aspect of the invention there is provided a process for preparing a pharmaceutical composition according to the invention comprising atovaquone or a pharmaceutically acceptable salt thereof having a D₉₀ particle size of greater than 3 to about 10 microns and further comprising one or more pharmaceutically acceptable excipients comprising admixing said atovaquone or a pharmaceutically acceptable salt thereof with one or more pharmaceutically acceptable excipients. The composition may be prepared by a process comprising wet or dry granulation techniques. Such a process may comprise:
i) admixing the particulate atovaquone or a pharmaceutically acceptable salt thereof with one or more pharmaceutical excipients;
ii) forming a wet granulation mixture;
iii) granulating the mixture;
iv) drying and sieving the resultant granules;
v) compressing the dried granules into tablet form; and
vi) optionally coating the tablet composition.

In a seventh aspect of the invention there is provided a process for preparing particulate atovaquone or a pharmaceutically acceptable salt thereof having a D₉₀ particle size of greater than 3 to about 10 microns comprising subjecting atovaquone or a pharmaceutically acceptable salt thereof to a technique selected from the group consisting comminution and de-agglomeration, micro-fluidisation, high pressure homogenisation and chemical treatment. In an embodiment the comminution techniques comprise grinding or milling in an air-jet mill or impact mill, a ball mill, vibration mill, mortar mill or pin mill. In further embodiments the chemical means comprises controlled precipitation or recrystallisation.

Of course it will be recognised by the skilled person that the compositions and atovaquone as disclosed herein lend themselves to a number of formulation types. For example controlled release compositions are within the scope of the invention. Such controlled-release compositions may comprise extended-release, sustained-release, delayed-release or modified-release. Further embodiments may also comprise multi-phasic release compositions wherein a proportion of the atovaquone is released immediately and release of the remainder is delayed. In still further embodiments, the composition may comprise additional API's with differing release kinetics.

### DETAILED DESCRIPTION OF INVENTION

The present invention provides atovaquone or a pharmaceutically acceptable salt thereof have a D₉₀ particle size diameter of between greater than 3 to about 10 microns. In order to produce atovaquone particles, e.g. crystals having the desired particle size and particle size distribution, conventional comminution and de-agglomeration techniques may be used, for example grinding in an air-jet mill or impact mill, a ball mill, vibration mill, mortar mill or pin mill. Further techniques such as micro-fluidisation can also be used. Chemical techniques such as controlled precipitation and/or recrystallisation may also be employed.

The known particle size analysis methods are suitable for determining the median particle size, for example particle size measurement using light, for example light-scattering methods or turbidimetric methods, sedimentation methods, for example pipette analysis using an Andreassen pipette, sedimentation scales, photosedimentometers or sedimentation in a centrifugal force field, pulse methods, for example using a Coulter counter, or sorting by means of gravitational or centrifugal force. Those methods are described, inter alia, in Voigt, loc. cit., pages 64-79.

The composition according to the invention may contain pharmaceutically acceptable excipients commonly used in pharmaceutical compositions, e.g. for oral administration.

According to one form of the present invention, the composition may be in the form of a tablet which comprises, a tablet core comprising a therapeutically effective dose of the atovaquone or a pharmaceutically acceptable salt thereof, optionally in a finely ground form, having a D₉₀ particle size of from greater than 3 to about 10 μm, preferably greater than 3 to about 5 μm, most preferably 4 to 5 μm and further excipients that are suitable for the manufacture of the compositions according to the invention. Alternatively, the D₉₀ particle size diameter is about 6 to about 8 microns.

One possible composition according to the present invention comprises a tablet composition. Such tablets according to the present invention comprise atovaquone or a pharmaceutically acceptable salt thereof of previously defined particle size and as such may be formulated into dosage forms, e.g. solid oral dosage forms such as tablets, with relative ease. Furthermore, such a particle size may also be beneficial in improving the bioavailability of atovaquone whilst still avoiding the problems that can be associated with fine particle sizes that are disclosed in the prior art. Still further the compositions meet all customary requirements, such as storage stability and colour stability.

Tablets according to the invention may be manufactured by any means at the disposal of the skilled practitioner. Commonly used means include compressing atovaquone with conventional tabletting excipients to form a tablet core using conventional tabletting processes. Optionally the tablet cores may be coated. Coatings may comprise one or more of modified release coatings, coatings that effect the release kinetics of atovaquone and conventional immediate release coatings for example the Opadry® series of aqueous film-coatings systems manufactured by Colorcon.

The tablet cores may be produced using conventional methods known in the art for example granulation methods, such as wet or dry granulation, with optional comminution of the granules and with subsequent compression and coating. Granulation methods are described, for example, in Voigt, loc. cit., pages 156-169.

Suitable excipients for the production of granules are, for example pulverulent fillers optionally having flow-conditioning properties, for example talcum, silicon dioxide, for example synthetic amorphous anhydrous silica acid of the Syloid® X type (Grace), for example SYLOID® 244 FP, microcrystalline cellulose, for example the Avicel® types (FMC Corp.) such as AVICEL® PH101, 102, 105, RC581 or RC 591, Emcocel® type (Mendell Corp.) or Elcema type (Degussa); carbohydrates, such as sugars, sugar alcohols, starches or starch derivatives, for example saccharose, lactose, dextrose, glucose, sorbitol, mannitol, xylitol, potato starch, maize starch, rice starch, wheat starch or amylopectin, tricalcium phosphate, calcium hydrogen phosphate or magnesium trisilicate; particularly preferred is microcrystalline cellulose; binders, such as gelatin, tragacanth, agar, alginic acid, cellulose ethers, for example methylcellulose, carboxymethylcellulose or hydroxypropyl methylcellulose, polyethylene glycols or ethylene oxide homopolymers, especially having a degree of polymerisation of approximately from 2.0×10³ to 1.0×10⁵ and an approximate molecular weight of about from 1.0×10⁵ to 5.0×10⁶, for example excipients known by the name Polyoxe® (Union Carbide), polyvinylpyrrolidone or povidones, and also agar or gelatine, the particularly preferred binder is hydroxypropyl methylcellulose such as Hypromellose; surface-active substances, for example anionic surfactants of the alkyl sulphate type, for example sodium, potassium or magnesium n-dodecyl sulphate, n-tetradecyl sulphate, n-hexadecyl sulphate or n-octadecyl sulphate, of the alkyl ether sulphate type, for example sodium, potassium or magnesium n-dodecyloxyethyl sulphate, n-tetradecyloxyethyl sulphate, n-hexadecyloxyethyl sulphate or n-octadecyloxyethyl sulphate, or of the alkanesulfonate type, for example sodium, potassium or magnesium n-dodecanesulfonate, n-tetradecanesulfonate, n-hexadecanesulfonate or n-octadecanesulfonate, or dioctyl sodium sulfosuccinate, or non-ionic surfactants of the fatty acid polyhydroxy alcohol ester type, such as sorbitan monolaurate, monooleate, monostearate or monopalmitate, sorbitan tristearate or trioleate, polyoxyethylene adducts of fatty acid polyhydroxy alcohol esters, such as polyoxyethylene sorbitan monolaurate, monooleate, monostearate, monopalmitate, tristearate or trioleate, polyethylene glycol fatty acid esters, such as polyoxyethyl stearate, polyethylene glycol 400 stearate, polyethylene glycol 2000 stearate, especially ethylene oxide/propylene oxide block polymers of the Pluronics® (BWC) or Synperonic® (ICI) type.

Granules may be produced in a manner known per se, for example using wet granulation methods known for the production of “built-up” granules or “broken-down” granules.

Methods for the formation of built-up granules may operate continuously and comprise, for example simultaneously spraying the granulation mass with granulation solution and drying, for example in a drum granulator, in pan granulators, on disc granulators, in a fluidised bed, by spray-drying or spray-solidifying, or operate discontinuously, for example in a fluidised bed, in a batch mixer or in a spray-drying drum.

Methods for the production of broken-down granules, which may be carried out discontinuously and in which the granulation mass first forms a wet aggregate with the granulation solution, which aggregate is then comminuted or formed into granules of the desired particle size and the granules then being dried. Suitable equipment for the granulation step are planetary mixers, low and high shear mixers, wet granulation equipment including extruders and spheronisers include, for example, apparatus from the companies Ligue, Glatt, Diosna, Fielder, Collette, Alexanderwerk, Ytron, Werner & Pfleiderer, Fuji, Nica, Caleva and Gabler.

The granulation mass consists of comminuted, preferably ground, atovaquone or a pharmaceutically acceptable salt thereof and the excipients mentioned above, for example pulverulent fillers, such as microcrystalline cellulose of the AVICEL® (FMC Corporation) type. AVICEL® PH 102 is especially suitable, or wetting agents/surfactants. Sodium lauryl sulphate or alternatively polysorbates such as Tween® (ICI), are particularly preferred surfactants. Depending on the method used, the granulation mass may be in the form of a premix or may be obtained by mixing the atovaquone into one or more excipients or mixing the excipients into the atovaquone. The wet granules are preferably dried, for example in the described manner by tray drying in an oven or drying in a fluidised bed dryer.

According to an alternative process variant, tablet cores are produced using the so-called compacting or dry granulation method in which the active ingredient is compressed with the excipients to form relatively large mouldings, for example slugs or ribbons, which are comminuted by grinding, and the ground material is compressed to form tablet cores.

A further alternative is that the granules are made by fluid bed granulation techniques either by spraying drug containing liquid onto small carrier particles or by forming the particles from the liquid and building upon them in subsequent passes through the fluid bed apparatus.

Suitable excipients for the compacting method are optionally those which are suitable for the conventional direct compression methods, for example dry binders, such as starches, for example potato, wheat and maize starch, microcrystalline cellulose, for example commercial products available under the trademarks Avicel® (FMC Corporation), Filtrak™, Hewetene® or Pharmacel®, highly dispersed silicon dioxide, for example Aerosil® (Degussa GmbH), mannitol, lactose, and also polyethylene glycol, especially having a molecular weight of from 4000 to 6000, cross-linked polyvinylpyrrolidone (Polyplasdones XL or Kollidon® CL by BASF AG), cross-linked carboxymethyl-cellulose (Acdisol® X CMC-XL by FMC Corp), carboxymethyl-cellulose [Nymcel, for example ZSB-10, (Nyma)], hydroxy-propyl methylcellulose, for example the quality HPMC 603, carboxymethyl starch <RTI [Explotab® X (Mendell) or Primojel® (Scholtens)], microcrystalline cellulose, for example Avicel® PH 102, dicalcium phosphate, for example Emcompress® (J Rettenmaier & Sohne GmbH & Co KG) or talcum. The addition of small amounts of, for example, lubricants, such as magnesium stearate, is also advantageous.

Compression to form tablet cores may be carried out in conventional tabletting machines, for example EK-0 Korsch eccentric tabletting machines or rotary tabletting machines such as Courtoy, Killian and Manesty Unipress. The tablet cores may be of various shapes, for example round, oval, oblong, cylindrical and various sizes, depending on the amount of atovaquone.

Controlled-release systems such as extended-release and sustained-release can be achieved by incorporating the active substance into a polymer matrix whereby the polymer acts to impede and/or control the flow of moisture and/or dissolved active substance within the dosage form. Control of the release of the active substance is believed to be achieved by the polymer swelling and reducing the rate of the passage of fluid. The drug then dissolves and drug laden fluid migrates back through the polymer and/or the polymer slowly erodes to release the drug into the body. This dosage form erosion also acts to present new areas of polymer/drug mixture that has not previously been exposed to the environment (ie. gastrointestinal system and corresponding fluids, etc). Polymers suitable to act as release controlling agents in a matrix system include cellulosic polymers such as HPMC K100MCR, HPMC K100, HPMC phthalate, ethylcellulose, cellulose acetate phthalate; Eudragit® polymers such as RL, RS, NM30D, NE30D, NE40D, FS30D; a mixture of polyvinylacetate and polyvinylalcohol such as Kollidon® SR (BASF AG); polyvinyl acetate phthalate; alginates such as propylene glycol alginate, sodium alginate, alginic acid; or any combination thereof. A non-polymeric material that can be utilised to effect the rate of release of the active pharmaceutical substance in a matrix system, either solely or in combination with a polymeric material is hydrogenated vegetable oil.

Additionally, a polymer system can be applied as a coating to compressed tablet cores or granules in order to provide a controlling layer thereon, either separately or in combination with a matrix control system. Controlled-release coating systems include the use of water-impermeable polymer coatings having a hole through the coating layer in combination with an osmotic pump system such as the OROS® technology (ALZA Corp); delayed-release or enteric coating systems such as cellulosic polymers such as hydroxypropyl methylcellulose (HPMC) lower viscosity grades, HPMC K100MCR, HPMC K100, HPMC acetyl succinate, HPMC phthalate, ethylcellulose, cellulose acetyl phthalate; Eudragit® polymers (Rohm GmbH) such as RL, RS, NM30D, NE30D, NE40D, L100-55, L30D-55, FS30D; a mixture of polyvinylacetate and polyvinylalcohol such as Kollidon® SR (BASF AG); polyvinyl acetate phthalate; or combinations thereof.

Any coating system may also require the use of a protective and/or separating subcoat(s). This can be achieved by applying a layer of polyvinylacetate; polyvinylpyrrolidone based products such as povidone, copovidone; a polyvinyl alcohol—polyethylene glycol graft polymer known as Kollidon® IR (BASF AG); cellulosic coating systems such as HPMC, Opadry® or Opadry® II systems (Colorcon); talc; sugars such sucrose, dextrose, lactose and the like; or other inert pharmaceutically acceptable excipient(s).

The pharmaceutical composition of this invention may additionally include at least one antiprotozoal agent selected from one of the following: proguanil, cycloguanil, mefloquinine, quinine, amodiaquine, chloroquine, hydroxychloroquine, pamaquine, primaquine, pyrimethamine, artemisinin, artemether, artesunate, artenimol, artemotil, halofantrine, lumefantrine or a pharmaceutically acceptable salt thereof. The preferred antiprotozoal agent is proguanil and more preferably proguanil hydrochloride. The amount of proguanil hydrochloride included can be between 20 and 250 mg, more preferably 25 to 100 mg.

The amount of atovaquone or a pharmaceutically acceptable salt thereof in a pharmaceutical composition according to the present invention is between 50 and 500 mg, preferably 62.5 and 250 mg.

### EXAMPLES

The following Examples illustrate the invention, but in no way limit the scope of the invention. Further, the atovaquone in the examples below has been micronised using standard techniques known in the art and as described above to a particle size with a D₉₀ of between greater than 3 to about 10 microns. Preferably the D₉₀ particle size is greater than 3 to about 5 microns, more preferably about 4 to about 5 microns. Alternatively, the D₉₀ particle size is about 6 to about 8 microns.

### Example 1

The particle size of atovaquone was determined by a Malvern Mastersizer as 30 mg in 900 mL of purified water with 3 drops of polysorbate 80, 10 minutes of sonication and a stirrer speed of 400 rpm.

The D₉₀ results are shown in Table 1:

TABLE 1

| Stir Time | | | | |
|---|---|---|---|---|
| (mins) | Run 1 | Run 2 | Run 3 | Run 4 |
| 1 | 5.088 | 5.182 | 5.049 | 5.283 |
| 2 | 5.089 | 5.183 | 5.049 | 5.282 |
| 3 | 5.090 | 5.182 | 5.048 | 5.283 |
| 4 | 5.088 | 5.183 | 5.047 | 5.284 |
| 5 | 5.088 | 5.184 | 5.048 | 5.281 |

### Example 2

The particle size of atovaquone was determined by a Malvern Mastersizer as 30 mg in 900 mL of purified water with 3 drops of polysorbate 80, 10 minutes of sonication and a stirrer speed of 400 rpm.

The D₉₀ results are shown in Table 2:

TABLE 2

| Stir Time | | | | |
|---|---|---|---|---|
| (mins) | Run 1 | Run 2 | Run 3 | Run 4 |
| 1 | 6.177 | 6.258 | 6.338 | 6.572 |
| 2 | 6.176 | 6.257 | 6.339 | 6.572 |
| 3 | 6.175 | 6.258 | 6.336 | 6.572 |
| 4 | 6.176 | 6.260 | 6.339 | 6.569 |
| 5 | 6.176 | 6.259 | 6.337 | 6.571 |

### Example 3

The particle size of atovaquone was determined by a Malvern Mastersizer as 30 mg in 900 mL of purified water with 3 drops of polysorbate 80, 10 minutes of sonication and a stirrer speed of 400 rpm.

The D₉₀ results are shown in Table 3:

TABLE 3

| Stir Time | | | | |
|---|---|---|---|---|
| (mins) | Run 1 | Run 2 | Run 3 | Run 4 |
| 1 | 4.280 | 4.260 | 4.124 | 4.285 |
| 2 | 4.280 | 4.260 | 4.124 | 4.285 |
| 3 | 4.280 | 4.261 | 4.123 | 4.286 |
| 4 | 4.280 | 4.261 | 4.123 | 4.286 |
| 5 | 4.280 | 4.260 | 4.125 | 4.286 |

### Example 4

The particle size of atovaquone was determined by a Malvern Mastersizer as 30 mg in 900 mL of purified water with 3 drops of polysorbate 80, 10 minutes of sonication and a stirrer speed of 400 rpm.

The D₉₀ results are shown in Table 4:

TABLE 4

| Stir Time | | | | |
|---|---|---|---|---|
| (mins) | Run 1 | Run 2 | Run 3 | Run 4 |
| 1 | 4.651 | 4.628 | 4.836 | 4.814 |
| 2 | 4.652 | 4.628 | 4.836 | 4.814 |
| 3 | 4.652 | 4.629 | 4.837 | 4.815 |
| 4 | 4.653 | 4.631 | 4.836 | 4.814 |
| 5 | 4.651 | 4.630 | 4.984 | 4.814 |

### Example 5

The particle size of atovaquone was determined by a Malvern Mastersizer as 30 mg in 900 mL of purified water with 3 drops of polysorbate 80, 10 minutes of sonication and a stirrer speed of 400 rpm.

The D₉₀ results are shown in Table 5:

TABLE 5

| Stir Time | | | |
|---|---|---|---|
| (mins) | Run 1 | Run 2 | Run 3 |
| 1 | 7.971 | 7.726 | 7.978 |
| 2 | 7.949 | 7.723 | 7.974 |
| 3 | 8.215 | 7.726 | 7.977 |
| 4 | 8.276 | 7.727 | 7.979 |
| 5 | 7.948 | 7.723 | 7.973 |

### Example 6

The ingredients of the pharmaceutical composition according to the invention can be prepared in accordance with acceptable pharmaceutical manufacturing practices. Preferably the manufacturing process will comprise wet granulation for example as described above, because of the amount of active pharmaceutical ingredient (API) required and also the lower compressibility of material at the preferred particle size.

An exemplary composition according to the invention is shown in Table 6.

A 250 mg/100 mg atovaquone/proguanil HCl composition containing atovaquone from Example 3 was prepared according to the following:

TABLE 6

| Ingredient | Amount/Tablet(mg) |
|---|---|
| Atovaquone | 250 |
| Proguanil HCl | 100 |
| Poloxamer ® 188 | 15 |
| Povidone K90 | 32 |
| Crospovidone | 120 |
| Water | qs |
| Microcrystalline Cellulose 102 | 51 |
| Magnesium stearate | 6 |
| Total | 574 |

The resultant compressed tablet may be further coated with, for example, an Opadry colour coat to about 3% weight gain.

### Example 7

A 62.5 mg/25 mg atovaquone/proguanil HCl composition containing atovaquone from Examples 3 and 4 was prepared according to the following:

TABLE 7

| Ingredient | Amount/Tablet(mg) |
|---|---|
| Atovaquone | 62.5 |
| Proguanil HCl | 25 |
| Poloxamer ® 188 | 3.75 |
| Povidone K90 | 8 |
| Crospovidone | 30 |
| Water | qs |
| Microcrystalline Cellulose 102 | 12.75 |
| Magnesium stearate | 1.5 |
| Total | 143.5 |

The resultant compressed tablet may be further coated with, for example, an Opadry colour coat to about 3% weight gain.

### Example 8

A 250 mg atovaquone composition containing atovaquone from Example 4 was prepared according to the following:

TABLE 8

| Ingredient | Amount/Tablet(mg) |
|---|---|
| Atovaquone | 250 |
| Poloxamer ® 188 | 15 |
| Povidone K90 | 32 |
| Crospovidone | 120 |
| Ethanol & water | qs |
| Microcrystalline Cellulose 102 | 51 |
| Magnesium stearate | 6 |
| Total | 474 |

### Example 9

Two bioequivalency studies were performed based on the pharmaceutical composition of example 6 against the proprietary product (reference listed drug by the FDA) under fed and fasted conditions. The studies were each double cross-over, blind studies involving 36 subjects. Each study was performed with 2 arms. In each arm, the subjects were allocated either the test or reference product and were dosed accordingly. At specific time intervals, blood samples were taken from each patient. The second arm was performed after a suitable wash-out period whereby the subjects were then dosed with the other product from the first arm. Again, blood samples were taken at the same time intervals.

All blood samples were then analysed to determine the maximum concentration achieved (Cₘₐₓ), the area under the curve from time zero (0) to last sample (AUC₀₋ₜ), and the area under the curve from time zero (0) to infinity (AUC_{0-∞}). Statistical analysis was performed to determine the 90% confidence interval (90% CI) for the logarithmically transformed ratio of test to reference averages for each parameter. The FDA and other health authorities set out statistical limits that define acceptable bioequivalency. In the US, this BE limit is a 90% CI range of 80% to 125%. The results for the fed and fasted bioequivalence studies are set out below in tables 9 and 10, respectively.

TABLE 9
Statistical Analysis for Atovaquone -
Fed Study

90% CI

| Parameter | Range | Mean |
|---|---|---|
| Cₘₐₓ | 92.6-109.0 | 100.3 |
| AUC₀₋ₜ | 87.4-107.2 | 97.3 |
| AUC_{0-∞} | 82.8-106.0 | 94.4 |

TABLE 10
Statistical Analysis for Atovaquone -
Fasted Study

90% CI

| Parameter | Range | Mean |
|---|---|---|
| Cₘₐₓ | 88.22-116.85 | 101.53 |
| AUC₀₋ₜ | 88.13-117.47 | 101.75 |
| AUC_{0-∞} | 88.40-116.45 | 101.46 |

Of course it will be apparent to one skilled in the art that the above compositions can be modified as required for example by the inclusion of colorants or taste enhancers and/or the application of a coating.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

It is to be understood that, if any prior art publication is referred to herein, such reference does not constitute an admission that the publication forms a part of the common general knowledge in the art, in Australia or any other country.

1. Atovaquone or a pharmaceutically acceptable salt thereof having a particle size diameter range with a D₉₀ of between greater than 3 to about 10 μm.

2. Atovaquone as claimed in claim 1 wherein the D₉₀ is between greater than 3 to about 5 μm.

3. Atovaquone as claimed in claim 1 wherein the D₉₀ is between about 4 to about 5 μm.

4. Atovaquone as claimed in claim 1 wherein the D₉₀ is between about 6 to about 8 μm.

5. A pharmaceutical composition comprising the atovaquone or a pharmaceutically acceptable salt thereof as claimed in claim 1 and at least one pharmaceutically acceptable excipient.

6. The pharmaceutical composition as claimed in claim 5 wherein the excipients are selected from Hypromellose, sodium lauryl sulfate, microcrystalline cellulose, sodium starch glycollate, poloxamer, Povidone, low substituted-hydroxypropyl cellulose, Crospovidone and magnesium stearate.

7. The pharmaceutical composition as claimed in claim 5 which further includes at least one antiprotozoal pharmaceutically active ingredient.

8. The pharmaceutical composition as claimed in claim 7 wherein the at least one antiprotozoal pharmaceutically active ingredient is selected from the group consisting of proguanil, cycloguanil, mefloquinine, quinine, amodiaquine, chloroquine, hydroxychloroquine, pamaquine, primaquine, pyrimethamine, artemisinin, artemether, artesunate, artenimol, artemotil, halofantrine and lumefantrine, or a pharmaceutically acceptable salt thereof.

9. The pharmaceutical composition as claimed in claim 7 wherein the at least one antiprotozoal pharmaceutically active ingredient is proguanil.

10. The pharmaceutical composition as claimed in claim 9 wherein the proguanil is proguanil hydrochloride.

11. The pharmaceutical composition as claimed in claim 5 wherein the amount of atovaquone is between 50 and 500 mg.

12. The pharmaceutical composition as claimed in claim 11 wherein the amount of atovaquone is between 62.5 and 250 mg.

13. The pharmaceutical composition as claimed in claim 5 wherein the amount of proguanil or pharmaceutically acceptable salt thereof is between 20 and 200 mg.

14. The pharmaceutical composition as claimed in claim 13 wherein the amount of proguanil or pharmaceutically acceptable salt thereof is between 25 and 100 mg.

15. Use of atovaquone or a pharmaceutically acceptable salt thereof as claimed in claim 1 in the manufacture of a medicament for the therapeutic and/or prophylactic treatment of malaria.

16. Use of atovaquone or a pharmaceutically acceptable salt thereof as claimed in claim 1 for the therapeutic and/or prophylactic treatment of malaria.

17. Use of a pharmaceutical composition as claimed in claim 5 in the therapeutic and/or prophylactic treatment of malaria.

18. A method for the therapeutic and/or prophylactic treatment of malaria comprising administering to a patient in need thereof, a therapeutically and/or prophylactically effective amount of the atovaquone as claimed in claim 1.

19. Process for preparing atovaquone as claimed in claim 1 comprising subjecting atovaquone particles to particle size reduction techniques.

20. Process for preparing a pharmaceutical composition of claim 5 by admixing atovaquone or a pharmaceutically acceptable salt thereof having a particle size diameter range with a D₉₀ of between greater than 3 to about 10 μm with at least one pharmaceutically acceptable excipient.

21. A method for the therapeutic and/or prophylactic treatment of malaria comprising administering to a patient in need thereof, a therapeutically and/or prophylactically effective amount of the pharmaceutical composition as claimed in claim 5.

22. The process of claim 19, comprising admixing the atovaquone particles with at least one pharmaceutically acceptable excipient.
